(19) United States
(12) Patent Application Publication
(10) Pub. No.: US 2022/0042067 A1
(43) Pub. Date: Feb. 10, 2022

(54) METHOD FOR ASSISTING PREDICTION OF SHUNT TROUBLE AND KIT THEREFOR

(71) Applicants: ASAHI KASEI PHARMA CORPORATION, Tokyo (JP); JAPAN COMMUNITY HEALTH CARE ORGANIZATION, Tokyo (JP)

(72) Inventors: Takeshi MATSUOKA, Tokyo (JP); Shigeru UEDA, Tokyo (JP); Satoru SANADA, Sendai-shi (JP); Manabu KANDA, Sendai-shi (JP); Hiroshi KITAMURA, Sendai-shi (JP)

(73) Assignees: ASAHI KASEI PHARMA CORPORATION, Tokyo (JP); JAPAN COMMUNITY HEALTH CARE ORGANIZATION, Tokyo (JP)

(21) Appl. No.: 17/281,400

(22) PCT Filed: Sep. 27, 2019
(86) PCT No.: PCT/JP2019/038209

§ 371 Date: Mar. 30, 2021

(30) Foreign Application Priority Data
Oct. 1, 2018 (JP) 2018-186932

(51) Int. Cl. C12Q 1/26

(52) U.S. Cl. CPC C12Q 1/26; G01N 2800/22

(57) ABSTRACT

The present invention provides a method for assisting a prediction of how likely a shunt trouble occurs, comprising a step of determining that the shunt trouble likely occurs, when a mevalonic acid concentration in a sample derived from a hemodialyzed patient is equal to or more than a preset cutoff value or increases with time.

### TECHNICAL FIELD

The present invention relates to a method for assisting a prediction of how likely a shunt trouble occurs using a mevalonic acid concentration in a sample derived from a hemodialyzed patient as an indicator and a kit therefor.

### BACKGROUND ART

For a practice of hemodialysis, blood must be drawn out of the body at a high flow of about 200 mL/min. For this reason, it is common to connect blood vessels from an artery to a vein thereby to increase a blood flow of the vein, followed by drawing blood from the vein to an extracorporeal circulation. Shunt means a blood vessel site at which an artery and a vein are connected. The dialysis having been practiced for a long period of time is known to cause blood clots in the blood vessel at a shunt site or a thickened inner side of a blood vessel thereby failing to obtain a blood flow required for dialysis, which results in the phenomenon called a shunt trouble.

### CITATION LIST

### Patent Literature

Patent Literature 1: International Publication No. WO2010/082665
Patent Literature 2: Japanese Patent Laid-Open No. 2013-158291

### Non Patent Literature

Non Patent Literature 1: J Vasc Access. 2017 Jul. 14; 18(4):295-300
Non Patent Literature 2: Clin J Am Soc Nephrol. 2010 August; 5(8):1447-50
Non Patent Literature 3: J Lipid Res. 2012 September; 53(9):1987-92

### SUMMARY OF INVENTION

### Technical Problem

The occurrence of a shunt trouble requires the stenosis removal by balloon dilation or the like at a stenotic lumen site of a shunt blood vessel or the formation of a new shunt which is an overwhelming burden for a hemodialyzed patient.

For the reduction of shunt trouble occurrence, statin treatment, as described in Non Patent Literature 1, was documented to have been effective in the reduction of a shunt trouble. However, Non Patent Literature 2 reports that the statin therapy is irrelevant to the reduction of a shunt trouble. Thus, the relation of the shunt trouble and statin and the occurrence mechanism of the shunt trouble are yet to be clarified whereby no effective prediction method has been documented up to date.

Assisting in a simple way a prediction of how likely a shunt trouble occurs and accordingly taking a preventive measure against it could lift the burden on a hemodialyzed patient.

### Solution to Problem

The present inventors formed a hypothesis that, in the occurrence mechanism of a shunt trouble, particularly shunt blood vessel stenosis, the activation of YAP (Yes-associated protein)/TAZ (transcriptional coactivator with PDZ-binding motif) plays a major role and the YAP/TAZ pathway and the mevalonate pathway present in the upstream thereof are involved. Further, the present inventors found that the occurrence of a shunt blood vessel stenosis can be predicted using a mevalonic acid concentration in serum of a hemodialyzed patient as an indicator, whereby the present invention was accomplished.

The present invention encompasses the following inventions.

[1]

A method for assisting a prediction of how likely a shunt trouble occurs, comprising a step of determining that the shunt trouble likely occurs, when a mevalonic acid concentration in a sample derived from a hemodialyzed patient is equal to or more than a preset cutoff value or increases with time.

[2]

The method according to [1], wherein the cutoff value is set at a value within a range from 4 to 9 ng/mL.

[3]

The method according to [2], wherein it is determined that how likely the shunt trouble occurs is at a frequency of more than once a year when the mevalonic acid concentration is 9 ng/mL or more.

[4]

The method according to [1], wherein the cutoff value is set at a value of 1.2 times or more a mevalonic acid concentration in a sample derived from a hemodialyzed patient without shunt trouble for 1 year or more.

[5]

The method according to any of [1] to [4], wherein the sample derived from the hemodialyzed patient is serum or plasma.

[6]

The method according to any of [1] to [5], wherein the mevalonic acid concentration is measured using an enzyme.

[7]

The method according to [6], wherein the enzyme is hydroxymethylglutaryl-CoA reductase.

[8]

The method according to [6] or [7], wherein the mevalonic acid concentration is measured using an enzyme cycling method.

[9]

A kit for assisting a prediction of how likely a shunt trouble occurs, comprising a reagent for measuring a mevalonic acid concentration in a sample derived from a hemodialyzed patient.

[10]

The kit according to [9], wherein the reagent comprises an enzyme.

[11]

The kit according to [10], wherein the enzyme is hydroxymethylglutaryl-CoA reductase.

[12]

A therapeutic method for prevention or delayed occurrence of a shunt trouble, comprising:

a step of determining that the shunt trouble likely occurs, when a mevalonic acid concentration in a sample derived from a hemodialyzed patient is equal to or more than a preset cutoff value or increases with time; and

a step of inhibiting signal transduction from the mevalonate pathway to the YAP/TAZ pathway in the patient diagnosed that the shun trouble likely occurs.

### Advantageous Effects of Invention

For the reduction of shunt trouble occurrence, statin treatment, as described in Non Patent Literature 1, was documented to have been effective in the reduction of a shunt trouble. However, Non Patent Literature 2 reports that the statin therapy is irrelevant to the reduction of a shunt trouble. Thus, the relation of the shunt trouble and the statin treatment has been controversial among researchers, however the present inventors had an idea that the statin treatment reduces a shunt trouble and consequently conceived as the mechanism of shunt trouble occurrence being that (1) bloodstream disturbance occurs in a shunt blood vessel, (2) the intima of the shunt blood vessel thickens by the disturbance, and (3) a shunt trouble from the thickened intima occurs, and further surmised and worked to verify that the YAP/TAZ activation is occurring in the vascular tissues before, after, or at the same time with, the above (2). Furthermore, the present inventors conceived that the activation of the mevalonate pathway is involved with the activation of YAP/TAZ and formed a hypothesis that an increase of a mevalonic acid amount, a rate-determining-step product of the mevalonate pathway, is associated with the shunt trouble occurrence however the accuracy of this hypothesis has been unknown.

Assuming the hypothesis by the present inventors holds true, a mevalonic acid concentration at a blood vessel site of shunt trouble occurrence in a hemodialyzed patient should be increasing, but in advance of the confirmation thereof, the present inventors surprisingly found the relation between the mevalonic acid concentration increase in a serum sample, but not at a blood vessel site of shunt trouble occurrence in a hemodialyzed patient and the shunt trouble occurrence, thereby enabling a prediction of shunt trouble occurrence by measuring a mevalonic acid concentration in a sample, particularly, blood, serum or plasma sample of a hemodialyzed patient, whereby the present invention was accomplished.

According to the present invention, the prediction of how likely a shunt trouble occurs can be assisted by using a mevalonic acid concentration in a sample of a hemodialyzed patient as an indicator and a suitable preventive therapeutic method can further be provided at an early stage. The measurement of a mevalonic acid concentration can be practiced highly sensitively and easily by using an enzyme cycling method.

### BRIEF DESCRIPTION OF DRAWINGS

FIG. 1 shows mevalonic acid concentrations (ng/mL) of 10 healthy subjects, 13 non-dialysis CKD patients, 13 patients without shunt trouble, and 64 patients with shunt troubles, and the error bars show standard deviations.

FIG. 2 shows mevalonic acid concentrations (ng/mL) of a group without shunt trouble occurrence, a group with the frequency of once a year or less of shunt trouble occurrence, and a group with the frequency of more than once a year of shunt trouble occurrence, and the error bars show standard deviations.

FIG. 3 shows the results of the relation between the total cholesterol and the shunt trouble occurrence with the presence or absence of the statin treatment.

### DESCRIPTION OF EMBODIMENTS

Hereinafter, the embodiments to carry out the present invention (hereinafter, referred to as the present embodiment) will be described below in detail. Note that the present invention is not limited to the following embodiments and can be carried out in various modifications within the scope of the gist.

The first embodiment provides a method for assisting a prediction of how likely a shunt trouble occurs, comprising a step of determining that the shunt trouble likely occurs, when a mevalonic acid concentration in a sample derived from a hemodialyzed patient is equal to or more than a preset cutoff value or increases with time.

Hemodialyzed patients (also simply referred to as “dialyzed patient”) means mammals such as a human who receives hemodialysis. Hemodialyzed patients can be hemodialyzed patients selected randomly. Alternatively, hemodialyzed patients can also be groups of hemodialyzed patients divided by sex, age, body weight, BMI, smoking history, drinking history, test numerical values such as serum triglyceride level, serum cholesterol level, and carotid thickening, medication history such as statin, the number of dialysis years, dialysis-induced diseases such as diabetes and IgA nephropathy, shunt formation sites, and shunt blood vessel types such as an autologous blood vessel or a synthetic graft.

The sample of a hemodialyzed patient in the present embodiment means any sample collected from a hemodialyzed patient. Examples of the sample from a hemodialyzed patient include, but not particularly intend to limit thereto, hair, body hair, nails, sweat, body fluids, tear, saliva, interdental fluid, saliva, blood, blood cells, serum, plasma, dialysate, blood vessel extracts, blood vessel-derived cells, organ extracts, and skin fragments. Of these, blood, serum, plasma, and dialysate can be easily collected and thus are preferable samples, with serum and plasma being particularly preferable.

Samples, when cannot be subjected to the mevalonic acid concentration measurement step immediately after collected, are preferably preserved. Sample preservation conditions for the mevalonic acid measurement are not particularly limited as long as the mevalonic acid concentration does not fluctuate from sample collection to sample measurement and, for example, such a concentration does not fluctuate 10% or more, preferably 5% or more, and more preferably 3% or more. For example, a preservation temperature can be, for example, 10 to 30° C. when preserved at room temperature, for example, 0 to 10° C. when refrigerated, and for example, −5 to −120° C. when frozen. Additionally, the preservation can be achieved with additives such as a preservative and a stabilizer so that the mevalonic acid concentration does not fluctuate.

The mevalonic acid concentration in a sample is presented by the weight or the number of moles of mevalonic acid present per unit weight or per unit volume of the sample. The mevalonic acid contained in a sample can be a case of including all forms such as a lactone form, a free acid form, and a salt form or a case where respective forms are separated, but a sample preferably includes all forms.

The mevalonic acid concentration in a sample referred herein includes not only a mevalonic acid amount per unit weight or per unit volume of the sample as described earlier but also a mevalonic acid amount corrected by amounts of other substances. The mevalonic acid concentration, when a sample is, for example, blood, serum, and plasma, also includes a mevalonic acid concentration corrected by dividing a mevalonic acid concentration in a sample by, for example, an albumin concentration or a glucose concentration or a sodium concentration singly or in combination in the sample. The mevalonic acid concentration, for example, when a sample is a red blood cell extract, also includes a mevalonic acid concentration corrected by dividing a mevalonic acid concentration in the extract by, for example, a hemoglobin concentration. The mevalonic acid concentration, for example, when a sample is a vascular tissue extract, also includes a mevalonic acid concentration corrected by dividing a mevalonic acid concentration in the extract by, for example, the total protein concentration in the extract.

The method for measuring a mevalonic acid concentration is not particularly limited as long as a method can measure a mevalonic acid concentration in a sample derived from a dialysis patient and examples thereof include LC-MS method, GC-MS method, immunoassay, enzyme method, radioisotope-based enzyme method, and enzyme cycling method. The enzyme cycling method described in Patent Literature 1 is easy and so highly sensitive that a mevalonic acid concentration in a sample derived from a dialysis patient can be measured, which is thus preferable as the measurement method for mevalonic acid concentration. Additionally, a use of a calibration sample described in Patent Literature 2 for the mevalonic acid concentration measurement by the enzyme cycling method described in Patent Literature 1 is preferable because of good operation procedure and accuracy. Examples of the enzyme used for the enzyme cycling method include hydroxymethylglutaryl-CoA reductase.

Examples of the reagent for the measurement of a mevalonic acid concentration include the reagents used for the enzyme cycling method described in Patent Literature 1 and Non Patent Literature 2.

Medical specialists have been substantially unable to diagnose how likely a shunt trouble occurs, but the method according to the present embodiment can assist a prediction of how likely a shunt trouble occurs in a hemodialyzed patient by using a mevalonic acid concentration in a sample of the hemodialyzed patient as an indicator. The medical specialist as used herein refers to a clinician with thorough knowledge of dialysis such as a nephrologist.

A shunt trouble means a phenomenon of failing to obtain a blood flow required for dialysis due to a thickened inner side of a blood vessel and the like and is diagnosed by, for example, i) a reduced blood flow from a shunt blood vessel during dialysis and an increased venous pressure of autotransfusion, ii) stenosis of a shunt blood vessel confirmed by angiography, ultrasound, or the like, iii) visual inspection and/or palpation by a medical specialist to confirm difficult conditions of hemostasis, or iv) a combination of i) to iii). Shunt stenosis can be diagnosed definitely using an imaging diagnosis and thus the step of ii) is the most important. The shunt trouble is also called a vascular access dysfunction or a vascular access failure.

A discrimination method for the presence of absence of a shunt trouble is well known by those skilled in the art, that is, among medical specialists, which is thus common understanding but is described specifically hereinbelow.

The reduced blood flow described in the above i) refers to a condition in which a blood flow is, for example, 90% or less or 75% or less than a blood flow when a shunt blood vessel was created or a blood flow during dialysis is, for example, 180 mL/min or less or 150 mL/min or less. The increased venous pressure of autotransfusion refers to, for example, 200 mmHg or more when a flow rate is 200 mL/min.

The stenosis of a shunt blood vessel described in the above ii) refers to, for example, a luminal diameter of a stenotic section being ½ or less or ⅓ or less than that of a non-stenotic section.

The prediction of how likely a shunt trouble occurs from a mevalonic acid concentration in a sample of a hemodialyzed patient refers to the decision whether a hemodialyzed patient without shunt trouble at the time of collecting a sample is likely to encounter a shunt trouble occurrence after a certain period of time. The certain period of time used herein is not particularly limited as long as it is a period during which a hemodialyzed patient has been continuing hemodialysis using the same shunt blood vessel but preferably, for example, 3 years, 2 years, 1 year, and a half year. Additionally, the frequency of test for predicting how likely a shunt trouble occurs by measuring a mevalonic acid concentration in a sample of a hemodialyzed patient can be suitably selected from viewpoints of QOL and medical economy of the hemodialyzed patient, such as every 3 months, every 6 months, or every 12 months.

The time of collecting a sample used herein is not particularly limited and includes, for example, a time-setting condition such as 8 o'clock in the morning and 1 o'clock in the afternoon, for example, a condition such as fasting time early in the morning, 8 hours or more, or 12 hours or more, after a meal, and for example, a condition under the starting time of dialysis and the ending time of dialysis.

The collection site, when a sample is blood, serum, and plasma, can be various sites and the collection can be made from, for example, a dialysis site, a blood vessel site in the upstream or downstream thereof, or an arm vein or a lower limb vein apart from a dialysis site, or sampled from a fingertip peripheral blood.

Additionally, when samples are collected from 2 or more sites, numerical values obtained by calculating such as dividing, taking differences, or averaging the mevalonic acid concentrations of these samples can also be used for a prediction of the occurrence of a shunt trouble.

The prediction of how likely a shunt trouble occurs can be carried out using a mevalonic acid concentration in a sample as an indicator. For example, when a mevalonic acid concentration is a certain concentration or more or increases with time, a shunt trouble is determined to likely occur. The prediction of how likely a shunt trouble occurs using a mevalonic acid concentration in a sample as an indicator can include the following Steps 1) to 3), which are however not intended to be limiting.

Step 1) Mevalonic acid concentrations in samples and the number of cases of shunt trouble occurrence and non-occurrence at a certain period of time after the sample obtention are measured.

Step 2) The data obtained in Step 1) are simply plotted or processed by ROC curve or the like thereby to set a mevalonic acid concentration at a certain concentration in the samples as a cutoff value.

Step 3) A mevalonic acid concentration equal to or more than the cutoff value in a sample leads to the prediction that a shunt trouble likely occurs.

The cutoff value in the above Step 2) when a sample is serum or plasma, can be set at, for example, 4 to 9 ng/mL, preferably 5 to 8 ng/mL, and more preferable 5.5 to 7.5 ng/mL. For example, when predicting how likely a shunt trouble occurs at the frequency of once a year or more as in Example 1, a cutoff value is set at 6.5 ng/mL and a mevalonic acid concentration of 6.5 ng/mL or more leads to the prediction that a shunt trouble likely occurs. Similarly, when predicting how likely a shunt trouble occurs at the frequency of more than once a year as in Example 2, a cutoff value can be set at 9 ng/mL. Setting specific cutoff values as described above enables an automatic conclusion of how likely a shunt trouble occurs, which has been difficult for medical specialists to conclude.

Alternatively, in addition to or in place of the above cutoff value as the indicator, how likely a shunt trouble occurs can also be predicted by the comparison with a mevalonic acid concentration in a sample derived from a hemodialyzed patient without shunt trouble for a year or more. For example, a hemodialyzed patient having a mevalonic acid concentration as high as 1.2 times or more, preferably 1.4 times or more, and more preferably 1.7 times or more the concentration of a dialyzed patient without shunt trouble for 1 year or more can be determined to likely have a shunt trouble.

The decision, which is made that a shunt trouble likely occurs through the above steps and any medical examination by medical specialists, enables the prescription of a therapeutic method for preventing or delaying the occurrence thereof.

The present inventors form a hypothesis that the YAP/TAZ pathway and the mevalonate pathway present in the upstream thereof are involved with the shunt trouble occurrence and conceive that any method inhibiting a signal transduction from the mevalonate pathway to the YAP/TAZ pathway can be a therapeutic method for preventing or delaying the occurrence of a shunt trouble. Thus, a patient diagnosed that a shunt trouble likely occurs can further be subjected to the therapeutic method including a step of inhibiting the signal transduction from the mevalonate pathway to the YAP/TAZ pathway.

The conceivable method inhibiting the signal transduction from the mevalonate pathway to the YAP/TAZ pathway include, for example, a prescription of an inhibitor of an enzyme which plays an important role in these pathways. Examples of such an enzyme include HMGCoA reductase, mevalonate kinase, phosphomevalonate kinase, diphosphomevalonate decarboxylase, farnesyl diphosphate synthase, and RhoA. The inhibitor of HMGCoA reductase is, for example, statin, and the inhibitor of farnesyl diphosphate synthase is, for example, bisphosphonate. The prescription of these inhibitors can be the preventive or therapeutic method.

The second embodiment provides a kit for assisting a prediction of how likely a shunt trouble occurs, including a reagent for measuring a mevalonic acid concentration in a sample derived from a hemodialyzed patient.

The kit is not particularly limited as long as it comprises a reagent for measuring a mevalonic acid concentration but, for example, a test kit capable of measuring using a commercial automated biochemical analyzer such as a Hitachi 7170S model is desirable.

The kit can be prepared at time of use but it is desirable to be preserved after produced under a refrigeration or freezing condition for a period of, for example, 1 week, 1 month, 3 months, 6 months, 1 year, and 2 years. The form of the kit can be a liquid reagent, a frozen reagent, lyophilized reagent, or a combination of these reagents. Further, the kit with modified stability, operability, measurement time, measurement accuracy and the like, can also be used.

Hereinafter, the present invention will be described furthermore specifically with reference to Examples and the like but the technical scope of the present invention is not limited to the following Examples and the like.

### Example 1

### <Relation Between the Mevalonic Acid Concentration and the Shunt Trouble Occurrence in Hemodialyzed Patients>

Serum samples were collected from 77 hemodialyzed patients starting from year 2014 to year 2017 and cryopreserved at −20° C. or −70° C. The 77 patients consist of a non-statin treatment group of 54 patients and a treatment group of 23 patients, and the profiles are shown in the following Table 1, Table 2, and Table 3.

TABLE 1

| | Non-statin | Statin |
|---|---|---|
| | treatment | treatment |
| Dialysis patient | group | group |
| Number of patients (n) | 54 | 23 |
| Age (y) | 68.9 ± 12.2 | 70.1 ± 11.2 |
| Male (%) | 63 | 47.8 |
| Body mass index (kg/m2) | 22.2 ± 5.0 | 22.5 ± 4.2 |
| Systolic blood pressure (mmHg) | 131.5 ± 21.5 | 141.4 ± 25.8 |
| Diastolic blood pressure (mmHg) | 71.4 ± 13.4 | 70.9 ± 12.8 |
| Diabetic Nephropathy: DMN (%) | 31.5 | 39.1 |
| Benign Nephrosclerosis: BNS (%) | 24.1 | 30.4 |
| Comorbidity (%) | | |
| Malignant tumor | 16.7 | 17.4 |
| Cardiovascular disease | 20.3 | 39.1 |
| Peripheral vascular disease | 7.4 | 4.4 |
| Cerebrovascular disease | 26 | 26.1 |
| Fistula site and type (%) | | |
| Radial artery - Subcutaneous | 74.1 | 73.9 |
| radiocephalic fistula using | | |
| autologous blood vessel (AVF) | | |
| Radial artery - Subcutaneous | 3.7 | 8.7 |
| brachiocephalic fistula using | | |
| autologous blood vessel (AVF) | | |
| Subcutaneous arteriovenous | 22.2 | 17.4 |
| graft using artificial blood | | |
| vessel (AVG) | | |

TABLE 2

Dialysis patient

| | Non-statin | Statin |
|---|---|---|
| | treatment | treatment |
| Drug compliance (%) | group | group |
| Antiplatelet agent | | |
| Aspirin | 25.9 | 47.8 |
| Clopidogrel | 7.4 | 34.8 |
| Others | 11.1 | 4.3 |
| Anticoagulant agent | | |
| Warfarin | 20.4 | 13 |
| Hypotensive agent | | |
| Calcium channel blocker | 44.4 | 60.9 |
| ACE-I/ARB | 33.3 | 73.9 |
| β-blocker | 27.8 | 17.4 |
| Antidiabetic agent | | |
| DPP-4 inhibitor | 18.5 | 47.8 |
| Insulin | 16.7 | 13 |

TABLE 3

| | Dialysis patient | |
|---|---|---|
| | Non-statin | Statin |
| | treatment | treatment |
| Test numerical value | group | group |
| Total protein (g/dL) | 7.0 ± 0.7 | 6.7 ± 0.7 |
| Albumin (g/dL) | 3.6 ± 0.5 | 3.6 ± 0.6 |
| Total cholesterol (mg/dL) | 180.3 ± 44.6 | 148.7 ± 31.5 |
| Triglyceride (mg/dL) | 127.0 ± 99.1 | 104.6 ± 55.8 |
| HDL-Cholesterol (mg/dL) | 48.6 ± 17.0 | 50.5 ± 19.3 |
| LDL-Cholesterol (mg/dL) | 103.8 ± 30.4 | 75.5 ± 21.4 |
| High sensitivity CRP (mg/dL) | 0.51 ± 0.79 | 0.78 ± 1.61 |
| Erythrocyte sedimentation rate (mm/h) | 39.3 ± 22.5 | 42.2 ± 23.7 |

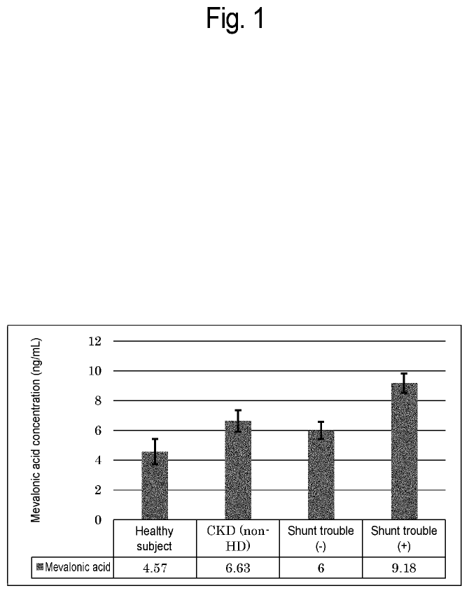

The measurement of mevalonic acid concentrations was carried out in year 2018 in accordance with the method described in Non Patent Literature 3. The hemodialyzed patients were divided into groups with a shunt trouble and without shunt trouble, and the relation of the grouping with the mevalonic acid concentration is shown in FIG. 1. The presence or absence of shunt trouble occurrence was established when medical specialists carried out angiography to the patients with a reduced shunt bloodstream or an increased venous pressure of autotransfusion recognized during actual dialysis and diagnosed as a shunt stenosis.

For reference, FIG. 1 also shows mevalonic acid concentrations of 10 healthy subjects and 13 non-dialysis CKD patients. FIG. 1 revealed that, in the dialysis patients, the shunt trouble occurrence group had a mevalonic acid concentration of 9.28 ng/mL which was significantly lower than a mevalonic acid concentration of 6 ng/mL of the shunt trouble non-occurrence group. A cutoff value set at 6.5 ng/mL, which is between 5.5 to 7.5 ng/mL, for predicting how likely a shunt trouble occurs reveals that the present method can be an effective test method. Additionally, FIG. 1 also reveals that the mevalonic acid concentration of non-dialysis CKD patients is 6.63 ng/mL and such a serum mevalonic acid concentration is significantly higher than the mevalonic acid concentration of 4.57 ng/mL of the healthy subjects. For the cutoff value, the larger the number of cases used for setting, the higher the accuracy a cutoff value can be set.

### Example 2

### <Relation Between the Mevalonic Acid Concentration and the Shunt Trouble Incidence in Hemodialyzed Patients>

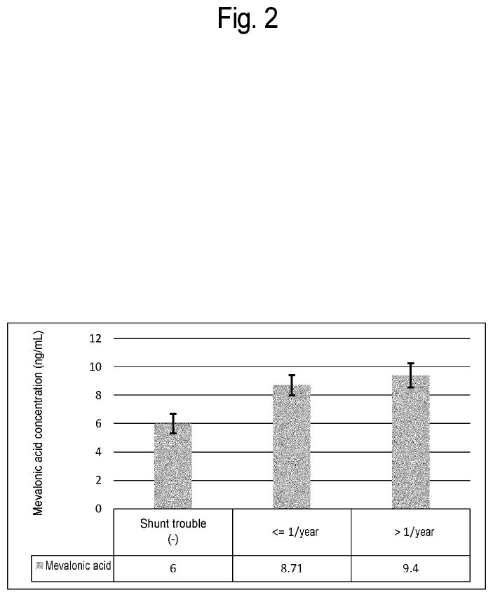

In Example 1, the 77 patients were divided into 3 groups based on the shunt trouble incidence of, in a period from year 2014 to year 2017, no occurrence, once a year or less, and more than once a year and the relation of the grouping with the mevalonic acid concentration is shown in FIG. 2.

According to FIG. 2, the group without shunt trouble occurrence has an average mevalonic acid concentration of 6 ng/mL, the group of once a year or less has 8.21 ng/mL, and the group of more than once a year has 9.4 ng/mL, revealing that the mevalonic acid concentrations are higher in the groups with higher shunt trouble occurrence frequencies. FIG. 2 reveals that when a cutoff value is set between 8.2 to 9.4 ng/mL, for example, 9.0 ng/mL, a method for testing a patient who likely has an occurrence of a shunt trouble can be provided. For the cutoff value in Example 2, the larger the number of cases used for setting, the higher the accuracy a cutoff value can be set as in Example 1.

### Reference Example 1

### <Relation Between the Total Cholesterol Concentration and the Shunt Trouble Occurrence in Hemodialyzed Patients>

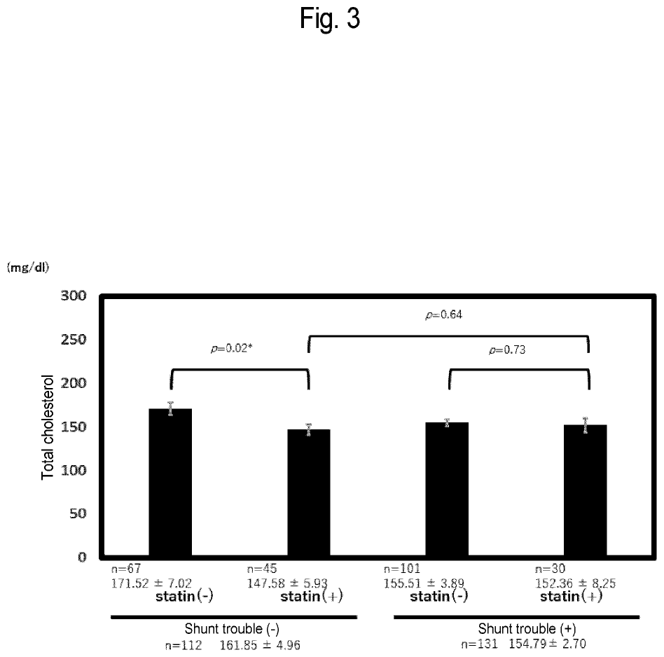

The mevalonic acid concentration is an indicator of cholesterol biosynthesis and thus, like a high mevalonic acid concentration, a high total cholesterol concentration was examined for the ability to predict shunt trouble occurrence. The hemodialyzed patients of Example 1 were divided into groups with a shunt trouble and without shunt trouble, and the results of examination on the relation of the grouping with the total cholesterol concentration are shown in Table 4 and Table 5. The group without shunt trouble had the total cholesterol concentration of 141.0±18.0 mg/dL (average of 8 patients), whereas the group with shunt trouble occurrence had 174.1±5.1 mg/dL (average of 69 patients). This result is, as with the mevalonic acid concentration, seemingly capable of predicting how likely a shunt trouble occurs due to the high cholesterol concentrations, however the relation with the total cholesterol concentration was further examined in detail by dividing the patients into a statin treatment group and a non-treatment group. As a result, in the non-statin treatment group, the group without shunt trouble occurrence had the total cholesterol concentration of 184.7±35.0 mg/dL (average of 3 patients), whereas the group with shunt trouble occurrence had 180.0±6.4 mg/dL (average of 51 patients), suggesting that having a high cholesterol concentration is irrelevant to the occurrence of a shunt trouble. Additionally, separate groups were investigated for the relation between the total cholesterol and the shunt trouble occurrence based on the presence or absence of statin treatment, and the results of which are shown in FIG. 3. These results failed to confirm, unlike the mevalonic acid concentration, the relation between a high total cholesterol concentration and the shunt trouble occurrence.

TABLE 4

| No shunt trouble | Total cholesterol | Mevalonic acid |
|---|---|---|
| occurred | (mg/dL) | (ng/ml) |
| Statin (−) n = 3 | 184.7 ± 35.0 | 6.87 ± 1.42 |
| Statin (+) n = 5 | 114.8 ± 9.0 | 4.68 ± 0.79 |
| Total n = 8 | 141.0 ± 18.0 | 5.50 ± 0.77 |

TABLE 5

| Shunt trouble | Total cholesterol | Mevalonic acid |
|---|---|---|
| occurred | (mg/dL) | (ng/ml) |
| Statin (−) n = 51 | 180.0 ± 6.4 | 9.55 ± 0.67 |
| Statin (+) n = 18 | 158.2 ± 6.7 | 8.16 ± 1.36 |
| Total n = 69 | 174.1 ± 5.1 | 9.19 ± 0.61 |

### INDUSTRIAL APPLICABILITY

According to the present invention, a prediction of how likely a shunt trouble occurs in a hemodialyzed patient can be assisted, and thus the diagnosis having been practiced by medical specialists becomes easier, whereby diagnosis of pathological conditions, prevention, and therapy relating to a shunt trouble can be efficient.

1. A method for assisting a prediction of how likely a shunt trouble occurs,
comprising a step of determining that the shunt trouble likely occurs, when a mevalonic acid concentration in a sample derived from a hemodialyzed patient is equal to or more than a preset cutoff value or increases with time.

2. The method according to claim 1, wherein the cutoff value is set at a value within a range from 4 to 9 ng/mL.

3. The method according to claim 2, wherein it is determined that how likely the shunt trouble occurs is at the frequency of more than once a year when the mevalonic acid concentration is 9 ng/mL or more.

4. The method according to claim 1, wherein the cutoff value is set at a value of 1.2 times or more a mevalonic acid concentration in a sample derived from a hemodialyzed patient without shunt trouble for 1 year or more.

5. The method according to claim 1, wherein the sample derived from the hemodialyzed patient is serum or plasma.

6. The method according to claim 1, wherein the mevalonic acid concentration is measured using an enzyme.

7. The method according to claim 6, wherein the enzyme is hydroxymethylglutaryl-CoA reductase.

8. The method according to claim 6, wherein the mevalonic acid concentration is measured using an enzyme cycling method.

9. A kit for assisting a prediction of how likely a shunt trouble occurs,
comprising a reagent for measuring a mevalonic acid concentration in a sample derived from a hemodialyzed patient.

10. The kit according to claim 9, wherein the reagent comprises an enzyme.

11. The kit according to claim 10, wherein the enzyme is hydroxymethylglutaryl-CoA reductase.

12. A therapeutic method for prevention or delayed occurrence of a shunt trouble, comprising:
a step of determining that the shunt trouble likely occurs, when a mevalonic acid concentration in a sample derived from a hemodialyzed patient is equal to or more than a preset cutoff value or increases with time; and
a step of inhibiting signal transduction from the mevalonate pathway to the YAP/TAZ pathway in the patient diagnosed that the shun trouble likely occurs.
